SEQUENCE LISTING (8 sequences)

SEQ ID NO: 1 - protein, 119 aa, synthetic construct
EVQLQQSGPELVKPGASVRISCKASGYIFTNYYIHWVKQRPGQGLEWIGWIYPGILNTKYNEVFKGKATLTADKSSSTAYMQLSSLSSEDSAVYFCARSDYRYDGFPYWGQGTLVTVSA

SEQ ID NO: 2 - protein, 109 aa, synthetic construct
DIVLTQSPVTLSVTPGESVSLSCRASQSISNNLHWYQQKSHESPRLLIKYASQSISGIPSRFSGSGSGTDFTLSLNSVETEDFGMYFCQQSNSWPLYTFGGGTKLEIRR

SEQ ID NO: 3 - protein, 8 aa, synthetic construct
GYIFTNYY

SEQ ID NO: 4 - protein, 8 aa, synthetic construct
IYPGILNT

SEQ ID NO: 5 - protein, 12 aa, synthetic construct
ARSDYRYDGFPY

SEQ ID NO: 6 - protein, 6 aa, synthetic construct
QSISNN

SEQ ID NO: 7 - skipped

SEQ ID NO: 8 - protein, 10 aa, synthetic construct
QQSNSWPLYT

(19) United States
(12) Patent Application Publication
(10) Pub. No.: US 2025/0382385 A1
(43) Pub. Date: Dec. 18, 2025

(54) ANTIVENOM MONOCLONAL ANTIBODIES OF RUSSELLS VIPER AND USES THEREOF

(71) Applicant: BUDDHIST TZU CHI MEDICAL FOUNDATION, Hualien (TW)

(72) Inventors: Li-Kuang CHEN, Hualien (TW); Ren-Chieh WU, Hualien (TW); Ping-Tse CHOU, Hualien (TW); Hui-Hua YANG, Hualien (TW)

(21) Appl. No.: 18/745,584

(22) Filed: Jun. 17, 2024

(51) Int. Cl. C07K 16/36; A61K 39/00

(52) U.S. Cl. CPC C07K 16/36; A61K 2039/505; C07K 2317/34; C07K 2317/76; C07K 2317/92

(57) ABSTRACT

Provided is an monoclonal antibody for treating envenomation of snake venom of Russell's viper. Also provided is an application of the monoclonal antibodies thereof, including a method for treating Russell's viper envenomation using the monoclonal antibodies.

### INCORPORATION BY REFERENCE OF A SEQUENCE LISTING XML

The present disclosure is being filed along with a Sequence Listing in electronic format. The Sequence Listing is provided as a file entitled 211543US-sequence listing, created on Jan. 9, 2024, which is about 7.14 KB in size. The contents in the electronic format of the Sequence Listing are incorporated by reference herein in their entirety.

### BACKGROUND

### 1. Technical Field

The present disclosure relates to a monoclonal antibody for treating envenomation of snake venom, in particular, a monoclonal antibody for treating snake venom of Russell's viper and a medical use thereof.

### 2. Description of Relevant Art

Russell's viper is distributed across South Asia and South East Asia, including India, Bangladesh, Thailand, Taiwan, etc. Russell's viper can be categorized into Daboia russelii mainly located in South Asia and Daboia siamensis mainly located in South East Asia. Russell's viper is highly toxic and has both neurotoxic and hemotoxic mix-type properties. Generally, the neurotoxins of Russell's viper is strongly venomous to small animals such as rodents and can quickly paralyzing small animals to facilitate predation. Large animals such as human beings are less effected by neurotoxins due to the size. The major injury from Russell's viper venom in large animals are caused by hemotoxin-induced coagulation symptoms, leading to intravascular blood clotting, resulting in thrombosis in small blood vessels of the limbs and/or organs. Simultaneously, systemic hemorrhage would occur as the coagulation factors are excessively depleted.

The major venom factors of Russell's viper's hemotoxins are Russell's viper venom factor-V (RVV-V) and Russell's viper venom factor-X (RVV-X), which are activation factors for human coagulation factor V and human coagulation factor X, respectively. By directly activating human coagulation factors V and X, RVV-V and RVV-X may induce cascade reaction of coagulation factors to produce clotting reaction, thereby causing thrombosis and tissue necrosis symptoms and the likes.

For Russell's viper envenomation, apart from the immediate procedure to remove venom entering the body through bitten, the most important procedure is to administer snake antiserum to the envenomed patient. However, current Russell's viper antiserum is a high titer serum obtained from horses immunized with the snake venom, which is expensive and have a lot of side effects. Therefore, development of better Russell's viper venom antidote is desperately needed.

Conventional serum obtained from immunized horses are polyclonal antibodies of snake venom antigen, the antibodies composition is complex and difficult to control. In 1975, Georges Kohler et al. developed a method of preparing monoclonal antibody using hybridoma. Through the isolation of monoclonal B cell and fusion with myeloma cell, it is possible to manufacture a monoclonal antibody that specifically bind to a single epitope. As the composition of monoclonal antibody is simple, the monoclonal antibody can more effectively target venom factors and treat the symptoms induced by venom factors, while reducing the risk of allergical and srerological reaction.

Based on the above, there is still an urgent need for a monoclonal antibody and a drug consist of the same that can replace snake antiserum in the art.

### SUMMARY

In view of the foregoing, the present disclosure provides an antivenom monoclonal antibody of Russell's viper or an antigen-binding moiety thereof specifically binding to a Russell's viper venom factor.

In one embodiment of the present disclosure, the monoclonal antibody or antigen-binding moieties thereof specifically binds to a RVV-V or a RVV-X, preferably the RVV-X.

In one embodiment of the present disclosure, the monoclonal antibody or the antigen-binding moiety thereof comprises a heavy chain variable region (VH) including three heavy chain complementarity determining regions (HCDR1, HCDR2 and HCDR3) and a light chain variable region (VL) including three light chain complementarity determining regions (LCDR1, LCDR2 and LCDR3).

In one embodiment of the present disclosure, the VH comprises an HCDR1 including an amino acid sequence of SEQ ID NO: 3, an HCDR2 including an amino acid sequence of SEQ ID NO: 4, and an HCDR3 including an amino acid sequence of SEQ ID NO: 5, and the VL comprises an LCDR1 including an amino acid sequence of SEQ ID NO: 6, an LCDR2 including an amino acid sequence of SEQ ID NO: 7, and an LCDR3 including an amino acid sequence of SEQ ID NO: 8.

In one embodiment of the present disclosure, the monoclonal antibody or the antigen-binding moiety thereof includes the VH including an amino acid sequence of SEQ ID NO: 1 or an amino acid sequence at least 85%, 90% or 95% identical to SEQ ID NO: 1. In another embodiment of the present disclosure, the monoclonal antibody or the antigen-binding moiety thereof includes the VL including an amino acid sequence of SEQ ID NO: 2 or an amino acid sequence at least 85%, 90% or 95% identical to SEQ ID NO: 2. In yet embodiment of the present disclosure, the monoclonal antibody or the antigen-binding moiety thereof includes the VH including an amino acid sequence of SEQ ID NO: 1 and the VL including an amino acid sequence of SEQ ID NO: 2.

The present disclosure further provides a polynucleotide encoding the monoclonal antibody or the antigen-binding moiety thereof according to at least one embodiment above, a vector including the polynucleotide, and/or a host cell including the vector. In one embodiment of the present disclosure, the vector is a eukaryotic expression vector, a prokaryotic expression vector or a viral vector. In another embodiment of the present disclosure, the host cell is any one selected from the group consisting of bacteria, yeast, insect cells, and mammalian cells. In yet another embodiment of the present disclosure, the host cell is any one selected from the group consisting of Escherichia coli, a Pichia genus, Spodoptera frugiperda cells, Chinese hamster ovary cells, and Human Embryonic Kidney Cells 293.

The present disclosure also provides a method of treating envenomation of snake venom of Russell's viper in a subject in need thereof. The method includes administering an effective amount of the monoclonal antibody or the antigen-binding moiety thereof according to at least one embodiment above to the subject.

In one embodiment of the present disclosure, the effective amount of the monoclonal antibody or the antigen-binding moiety thereof is in a range from 1 ng to 10 μg, preferably in a range from 18 ng to 1800 ng or in a range from 1 μg to 5 μg.

In one embodiment of the present disclosure, administration of the monoclonal antibody or the antigen-binding moiety thereof is intraperitoneal, intramuscular, intravenous or subcutaneous.

In one embodiment of the present disclosure, the method further includes administering a pharmaceutical acceptable excipient. In another embodiment of the present disclosure, the method includes administering a formulation including the monoclonal antibody or the antigen-binding moiety thereof according to at least one embodiment above to the subject and a pharmaceutical acceptable excipient.

In one embodiment of the present disclosure, the method further includes administering an additional antidote for Russell's viper, that is, administration of the monoclonal antibody or the antigen-binding moiety thereof is in combination with the additional antidote for Russell's viper. In another embodiment of the present disclosure, the additional antidote for Russell's viper includes a Russell's viper antiserum, a polyclonal Russell's viper antibody or a combination thereof.

The present disclosure provides a method of detecting or determining venom of Russell's viper in a sample, including contacting the monoclonal antibody or the antigen-binding moiety thereof according to at least one embodiment above with the sample. In one embodiment of the present application, the sample is a blood sample.

The present disclosure provides a kit for determining a concentration of venom of Russell's viper in a sample, and the kit includes 1) the monoclonal antibody or the antigen-binding moiety thereof according to at least one embodiment above; 2) a vessel; and 3) an instruction for using the monoclonal antibody or the antigen-binding moiety thereof to determine the concentration of venom of Russell's viper.

In one embodiment of the present disclosure, the sample is a blood sample.

The monoclonal antibody or the antigen-binding moiety thereof of the present disclosure can specifically bind to Russell's viper venom to achieve the treatment of Russell's viper envenomation and/or the detection of Russell's viper venom.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the following descriptions of the embodiments, with reference made to the accompanying drawings.

FIG. 1 is a graph illustrating the survival rate for detecting Median Lethal Dose (LD₅₀) by intraperitoneal injection of venom of Russell's viper to mice.

FIG. 2 is a graph showing the survival rate for detecting Median Thrombosis Dose (TD₅₀) by subcutaneous injection of venom of Russell's viper to paws of mice.

FIG. 3 is pictures showing the blackening of mice paws observed in detection of Median Thrombosis Dose (TD₅₀) mentioned above with different dosage of venom of Russell's viper.

FIGS. 4A-4E are Western blots of the monoclonal antibody of the present disclosure that specifically binds to venom of Daboia russelli formosensis (Formosan Russell's viper) (i.e., crude venom antigen) and purchased Daboia russelii pure RVV-X protein, which are identified as C column and P column, respectively.

FIGS. 5A-5E are Western blots for testing the binding between the monoclonal antibody of the present disclosure with denatured Formosan Russell's viper venom (i.e., crude venom antigen) and denatured purchased Daboia russelii pure RVV-X protein. C columns stand for Formosan Russell's viper venom, and P column stands for purchased pure RVV-X protein.

FIGS. 6A and 6B are Western blots of the monoclonal antibody 7-7C of the present disclosure that specifically bind to pure RVV-X protein separated from Formosan Russell's viper (Daboia siamensis) venom using a column.

FIG. 6A is a Western blotting test result of the monoclonal antibody 7-7C against column purified Formosan Russell's viper venom. FIG. 6B is a Western blotting test result of the monoclonal antibody 7-7C against denatured column purified Formosan Russell's viper venom.

FIG. 7 is a line graph indicating the changes of clotting time of clotting of recalcification of plasma in human beings caused by different concentrations of Formosan Russell's viper venom.

FIGS. 8A and 8B are line graphs showing changes of clotting time of the monoclonal antibodies 5-6B (mAb 5-6), 7-7C (mAb 7-7), 16-2F (mAb 16-2) and 23-9B (mAb 23-9) of the present disclosure in different concentrations on inhibition of clotting caused by Formosan Russell's viper venom (RVV).

FIG. 9 is a line graph showing ratios of the clotting time (CTᵥₑₙₒₘ) after adding Russell's viper venom and the clotting time (CTv_{enom+Ab}) after adding a mixture of Russell's viper venom and the monoclonal antibodies 5-6B (mAb 5-6), 7-7C (mAb 7-7), 16-2F (mAb 16-2) or 23-9B (mAb 23-9). Monoclonal antibody 9-5C (mAb 9-5) is used as a control group.

FIGS. 10A and 10B are line graphs showing S-2222 color development of the monoclonal antibodies 5-6B, 7-7C, 16-2F and 23-9B of the present disclosure each mixed with Russell's viper venom.

FIG. 11 is a graph showing the survival rate of mice injected intraperitoneally with Russell's viper venom in conjunction with Russell's viper venom antiserum or the monoclonal antibody 7-7C (Ab 7-7).

FIG. 12 is a graph showing the survival rate of mice injected intraperitoneally with Russell's viper venom in conjunction with different dosage combinations of Russell's viper venom antiserum and the monoclonal antibody 7-7C.

FIG. 13 is pictures of the blackening of mice paws injected with Russell's viper venom in conjunction with Russell's viper venom antiserum or the monoclonal antibody 7-7C (mAb 7-7).

FIG. 14 is pictures of the blackening of mice paws that was first intraperitoneally injected with Russell's viper venom antiserum and then injected with Russell's viper venom in conjunction with the monoclonal antibody 7-7C (mAb 7-7C).

FIG. 15 shows the amino acid sequences of the heavy chain variable region (VH) and light chain variable region (VL) of the monoclonal antibody 5-6B.

### DETAILED DESCRIPTION

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the following examples for carrying out this disclosure without contravening its spirit and scope, for different aspects and applications.

It should be further noted, the singular forms “a” and “the” used herein are intended to include plural referents unless they are otherwise specifically limited to a single referent. In addition, unless otherwise specifically indicated, the term “or” and “and/or” used herein are interchangeable.

The term “comprise/comprising,” “include/including,” “contain/containing” or “have/having” used herein refers to some elements such as components, steps and the likes that exist in a product, a method, use and the like of the present disclosure. Unless specifically indicated in the context otherwise, those undocumented and unspecified elements are also open to exist in a product, a method or use of the present disclosure, whether it is necessary or not. In other word, those undocumented and unspecified elements are not restrictively excluded.

The term “subject” used herein refers to animals, such as mammals, including, but not limited to, primates (e.g., humans), cattle, sheep, goats, horses, dogs, cats, rabbits, rats, mice, etc. In one embodiment of the present disclosure, the subject is a human.

The term “an effective amount of a medicament” used herein refers the quantity of the medicament which achieves therapeutic effect when the medicament is administered to a subject.

The term “treat/treating/treatment” as used herein refers to any indicator of success in the treatment or improvement of discomfort or symptoms (such as Russell's viper envenomation), including any objective or subjective parameter. Examples of indicator of success are alleviation, relief, reduction of symptoms; or make a symptom, injury, pathology, or condition more tolerable to a subject; reduce the frequency or duration of a symptom or an uncomfortable condition; or in some cases, prevent the onset of a symptom or an uncomfortable condition. Treatment or improvement of a symptom may be based on any objective or subjective parameter, including, for example, clotting time.

The term “about” used in the present disclosure refers to an error or a range of a numerical value, a numerical range or a ratio is within 20% of the numerical value, the numerical range or the ratio, preferably within 10%, more preferably to fluctuate within 5%. The quantitative values used herein are approximations, meaning that they can be inferred even when the term “about” is not used.

The numerical ranges described herein are inclusive and combinable, and any value that falls within the numerical ranges described herein can be used as a maximum or minimum value to derive a subrange. For example, the numerical range of “18 to 1800 ng” should be understood to include any subrange between the minimum value of 18 ng and the maximum value of 1800 ng, such as: 18 ng to 500 ng, 200 ng to 1800 ng and 100 ng to 1000 ng. In addition, various numerical endpoints described herein can be optionally selected as a maximum or minimum value to derive a numerical range. For example, a numeric range of 1 to 4 μg, 3 to 5 μg, or 2 to 4 μg can be derived from 1, 2, 3, 4, 5 μg.

The purpose of the present disclosure is to provide a monoclonal antibody against venom of Russell's viper or the antigen-binding moiety thereof.

The present disclosure provides a monoclonal antibody for venom of Russell's viper or the antigen-binding moiety thereof, and the monoclonal antibody or the antigen-binding moiety thereof can bind specifically to a RVV factor.

In one embodiment of the present disclosure, the Russell's viper venom factor is RVV-V or RVV-X. In an exemplary embodiment of the present disclosure, the Russell's viper venom factor is RVV-X.

In one embodiment of the present disclosure, the monoclonal antibody or the antigen-binding moiety thereof includes a heavy chain variable region (VH) including an amino acid sequence of SEQ ID NO: 1, and a light chain variable region (VL) including an amino acid sequence of SEQ ID NO: 2. In another embodiment of the present disclosure, the VH is an amino acid sequence of SEQ ID NO: 1, and the VL is an amino acid sequence of SEQ ID NO: 2.

the monoclonal antibody or the antigen-binding moiety thereof the VH has three heavy chain complementarity determining regions of HCDR1, HCDR2 and HCDR3, and the VL has three light chain complementarity determining regions of LCDR1, LCDR2 and LCDR3. In another embodiment of the present disclosure, the VH includes an HCDR1 including an amino acid sequence of SEQ ID NO: 3, an HCDR2 including an amino acid sequence of SEQ ID NO: 4, and an HCDR3 including an amino acid sequence of SEQ ID NO: 5, and the VL includes an LCDR1 including an amino acid sequence of SEQ ID NO: 6, an LCDR2 including an amino acid sequence of SEQ ID NO: 7, and an LCDR3 including an amino acid sequence of SEQ ID NO: 8. In yet another embodiment of the present disclosure, the VH includes an HCDR1 of an amino acid sequence of SEQ ID NO: 3, an HCDR2 of an amino acid sequence of SEQ ID NO: 4, and an HCDR3 of an amino acid sequence of SEQ ID NO: 5, and the VL includes an LCDR1 of an amino acid sequence of SEQ ID NO: 6, an LCDR2 of an amino acid sequence of SEQ ID NO: 7, and an LCDR3 of an amino acid sequence of SEQ ID NO: 8.

In one embodiment of the present disclosure, the amino acid sequence of the monoclonal antibody or the antigen-binding moiety thereof may be an amino acid sequence that is at least 85%, 90% or 95% identical to the aforementioned amino acid sequence. In another embodiment of the present disclosure, the amino acid sequence of the monoclonal antibody or the antigen-binding moiety thereof may have amino acid mutations that do not affect the specific binding ability of the monoclonal antibody or the antigen-binding moiety thereof, such as a conservative mutation. A conservative mutation is a substitution mutation of an amino acid molecule with another amino acid molecule that is similar in physical and chemical properties.

The present disclosure further provide a polynucleotide encoding the Russell's viper venom monoclonal antibody or the antigen-binding moiety thereof, a vector including the polynucleotide, and/or a host cell including the vector.

In one embodiment of the present disclosure, the host cell includes, but not limit to, bacteria, yeast, insect cells and mammalian cells. A person skilled in the art can select the appropriated cells for expressing a monoclonal antibody or the antigen-binding moiety thereof base on known art. Some exemplary host cells include Escherichia coli, Pichia yeast, Spodoptera frugiperda cells, Chinese hamster ovary cells, Human Embryonic Kidney Cells 293, etc.

The present disclosure also provides a method of treating Russell's viper envenomation by administering effective amount of the monoclonal antibody or the antigen-binding moiety thereof of the present disclosure to a subject in need thereof.

In one embodiment of the present disclosure, the effective amount of the monoclonal antibody or the antigen-binding moiety thereof of the present disclosure is 1 ng to 10 μg, such as about 1 ng, about 2 ng, about 3 ng, about 4 ng, about 5 ng, about 6 ng, about 7 ng, about 8 ng, about 9 ng, about 10 ng, about 11 ng, about 12 ng, about 13 ng, about 14 ng, about 15 ng, about 16 ng, about 17 ng, about 18 ng, about 19 ng, about 20 ng, about 30 ng, about 40 ng, about 50 ng, about 100 ng, about 200 ng, about 300 ng, about 400 ng, about 500 ng, about 600 ng, about 700 ng, about 800 ng, about 900 ng, about 1 μg, about 1.1 μg, about 1.2 μg, about 1.3 μg, about 1.4 μg, about 1.5 μg, about 1.6 μg, about 1.7 μg, about 1.8 μg, about 1.9 μg, about 2 μg, about 2.5 μg, about 3 μg, about 3.5 μg, about 4 μg, about 4.5 μg, about 5 μg, about 5.5 μg, about 6 μg, about 6.5 μg, about 7 μg, about 7.5 μg, about 8 μg, about 8.5 μg, about 9 μg, about 9.5 μg, or about 10 μg. In another embodiment of the present disclosure, the effective amount of the monoclonal antibody or the antigen-binding moiety thereof of the present disclosure is 18 ng to 1800 ng. In yet another embodiment of the present disclosure, the effective amount of the monoclonal antibody or the antigen-binding moiety thereof of the present disclosure is 1 μg to 5 μg. The various aforementioned numerical endpoints can be optionally selected as a maximum or minimum value to derive a numerical range.

In one embodiment of the present disclosure, the monoclonal antibody or the antigen-binding moiety thereof of the present disclosure is administered intraperitoneally, intramuscularly, intravenously or subcutaneously.

In one embodiment of the present disclosure, the monoclonal antibody or the antigen-binding moiety thereof of the present disclosure further includes a pharmaceutical acceptable excipient. In another embodiment of the present disclosure, the monoclonal antibody or the antigen-binding moiety thereof of the present disclosure can further includes other components such as pharmaceutically acceptable carriers, additives, adjuvants and/or excipients for injection. For example, the composition may include, but not limit to, any combination of one or more agent selected from the group consisting of solvents, solubilizers, stabilizers, tonicity enhancers, penetration enhancers, pH regulator, surfactants, buffers, preservatives, emulsifiers, suspending agents, and antimicrobial agents.

In one embodiment of the present disclosure, the monoclonal antibody or the antigen-binding moiety thereof of the present disclosure can be used in combination with other Russell's viper antidote for treatment of Russell's viper envenomation. In one embodiment of the present disclosure, the Russell's viper antidote that can be used in combination includes Russell's viper antiserum and polyclonal Russell's viper antibodies. Specific examples of Russell's viper antidote include, but not limit to, high titer serum obtained from horses immunized with the Russell's viper venom and Antivenin of D. siamensis (Lyophilized) (Taiwan Centers for Disease Control and Prevention, Taiwan).

The monoclonal antibody or the antigen-binding moiety thereof of the present disclosure can also be used to detect or determine the presence of venom of Russell's viper in a sample. The method includes contacting the monoclonal antibody or the antigen-binding moiety thereof of the present disclosure with the sample, and determining if the sample contains proteins that can be specifically bound with the monoclonal antibody or the antigen-binding moiety thereof of the present disclosure.

The present disclosure provides a kit for determining a concentration of venom of Russell's viper in a sample, and the kit includes 1) the monoclonal antibody or the antigen-binding moiety thereof of the present disclosure; 2) a container; and 3) an instruction for using the monoclonal antibody or the antigen-binding moiety thereof to determine the concentration of venom of Russell's viper.

In one embodiment of the present disclosure, the sample mentioned above is obtained from the subject suspected of being envenomed by Russell's viper, and then the method and/or the kit is used to confirm the concentration of Russell's viper venom in the blood sample ex vivo, so that a physician or specialist can determine whether the patient is envenomed and the severity of the envenomation.

Multiple examples have been used to illustrate the present disclosure. The examples below should not be taken as a limit to the scope of the present disclosure.

### Examples

### 1. Materials

Source of snake venom used in experiments:
Russell's viper venom (RVV; a crude snake venom): Supernatant collected from centrifuged Formosan Russell's viper venom independently acquired by the laboratory.
Russell's viper venom factor-X (RVV-X, also known as Russell's viper coagulation factor X activating enzyme): Purchased from Haematologic Technologies, Inc., product name is RVV-X Activator, and is a purified RVV-X at a concentration of 6.7 mg/mL.
Instrument:
Micro centrifuge: National Labnet CO.
Oscillator (vortex-2 genie): Scientific Industries, IMC., USA.
Shaker: Major Science 500 VP, Taiwan.
SNAP i.d.™ Protein Detection System: Millipore, Ireland, Advanced.
Hoefer™ Electrophoresis Unit: SE260, Amersham Bioscience.
Hoefer™ Transfer Unit: TE 22, Amersham Bioscience.
Power supply (electrophoresis power supply): Consort E802.
Precision thermal circulation oven (drying oven): Deng YNG D060
Antibody purification liquid chromatography system (ÄKTAprime): Amersham Pharmacia Biotech.
Enzyme immunoassay analyzer (microplate reader): Dynex Technologies The Microtiter® company.
Dry Bath Incubator: VH-01, Violet biosciences, Inc.
Optical spectrum analyzer: uQuant MQX200, Bio-Tek instruments, Inc., USA.
Multi-fluorescent nucleic acid quantitative analysis system (Fluorescent gel image system): Gene-Cam-Flexi, Taiwan.
Reaction block assembly (Alpha Unit™ Block Assembly for PTC DNA Engine™ Systems): PTC-2000, MJ-Resecrch, Inc., USA.
Freeze drying machine: RVT4104, Thermo.
Tissue homogenizer (Homogenizer): PRO200, The Lab Depot, Inc., USA.
Electrophoresis system: SE250/260, Amersham.
Internet resources:

The protein sequence analysis and comparison tools used in this experiment are RCSB Protein Data Bank (http://www.rcsb.org/pdb/home/home.do), ExPASy Proteomics Sever (http://expasy.org/), Protein Molecular Weight Calculator (http://www.sciencegateway.org/tools/proteinmw.htm) and Align Sequences using ClustalW2| EBI (http://www.ebi.ac.uk/Tools/msa/clustalw2/).

### 2. Method

### 5 Western Blot:

### (1) Experiment Materials

| | |
|---|---|
| 10% Acrylamide gel | Independently prepared |
| Sample buffer | Independently prepared |
| Tris-glycine SDS buffer | Independently prepared |
| (electrophoresis buffer) | |
| Methanol | J. T. Baker, Cat. 9093-68 |
| Transfer buffer | Independently prepared |
| Tris buffer saline (TBS) | Independently prepared |
| TBS with 0.1% Tween 20 | Independently prepared |
| PVDF transfer film | Pall Corporation BioTrace ™ |
| Primary antibodies | Monoclonal antibodies independently |
| | constructed by the laboratory |
| Secondary antibodies | Goat anti-mouse IgG alkaline |
| | phosphatase, AbD Serotec |
| Blocking reagent_{—} | 10% skim milk powder in 1X TBS |
| Goat serum | GIBCO ™ Invitrogen Corporation, |
| | USA |
| Alkaline phosphatase (AP) buffer | Independently prepared |
| Nitrotetrazolium Blue Chloride | Amresco ®, Cat. 0329 |
| (NBT) | |
| 5-Bromo-4-chloro-3- | Sigma, Cat.08851G |
| indolylphosphate | |
| (BCIP) p-toluidine salt | |

### (2) Various Independently Prepared Solutions

| | | |
|---|---|---|
| 10% Acrylamide separation gel | 15.0 | mL |
| Double-distilled water (ddH₂O) | 5.9 | mL |
| 30% Acrylamide mixture (Sigma, Cat. A3574) | 5.0 | mL |
| 4X 1.5M Tris buffer (pH 8.8) (J. T. Baker, Cat. 4109-6) | 3.8 | mL |
| 10% SDS (J. T. Baker, Cat. 4095-4) | 0.15 | mL |
| 10% APS (Sigma, Cat. A9164-100G) | 0.15 | mL |
| TEMED (Amresco ®, Cat. 0761) | 0.006 | mL |

### 4X Sample Loading Buffer

8% SDS (J. T. Baker, Cat. 4095-4)
0.4% Bromophenol blue (Sigma, Cat. B5525-G)
40% Glycerol (Merck, Cat. 64271)
20% 2-Mercaptoethanol (Amresco®, Cat. 0482)
20 mM Tris buffer (pH6.8) (J. T. Baker, Cat. 4109-6)
5X Tris-glycine electrophoresis buffer (pH8.3)
125 mMTris buffer (J. T. Baker, Cat. 4109-6)
1.25 M Glycine (J. T. Baker, Cat. 4059-02)

### Transfer Buffer

20 mM Tris-base (pH8.3) (J. T. Baker, Cat. 4109-6)
150 mM Glycine (J. T. Baker, Cat. 4059-02)
20% Methanol (J. T. Baker, Cat. 9093-68)
10X Tris Buffered Saline (TBS)
250 mM Tris base (J. T. Baker, Cat. 4109-6)
8% NaCl (J. T. Baker, Cat. 3624-05)
26.8 mM KCl (Riedel-dehaën®, Cat. 31248)
0.1% Tween 20 in Tris buffer saline
1X TBS (Amresco®, Cat. J640)
0.1% Tween 20 (Amresco®, Cat. 0777)
Alkaline phosphatase assay buffer
100 mM Tris base (pH9.5) (J. T. Baker, Cat. 4109-6)
100 mM NaCl (J. T. Baker, Cat. 3624-05)
5 mM MgCl 2 (J. T. Baker, Cat. 2444-01)
5X RIPA lysis buffer
250 mM Tris base (pH8.0) (J. T. Baker, Cat. 4109-6)
750 mM NaCl (J. T. Baker, Cat. 3624-05)
1% NP-40 (Sigma, Cat. I18896)
1% Sodium deoxycholate (Thermo, Cat. 89904)
0.1% SDS (J. T. Baker, Cat. 4095-4)

### (3) Preparation of Experiment Materials

60 μL of 4X sample loading buffer (independently prepared), 48 μL of RIPA buffer (independently prepared) and 30 μL of Cock Tail Protease inhibitor (CTPI, Roche) were loaded into an eppendorf, and 2 μL of Formosan Russell's viper venom and 192 μL of double-distilled water were added last. The mixture was vortexed to obtain the Russell's viper venom sample.

81 μL of 4X sample loading buffer (independently prepared), 27 μL of 2-mercaptoethanol (2-ME, Amresco®) and 30 μL of Cock Tail Protease inhibitor (CTPI, Roche) were loaded into an eppendorf, and 2 μL of Formosan Russell's viper venom and 192 μL of double-distilled water were added last. The mixture was vortexed and then placed into 37° C. drying oven for 30 minutes to obtain the Russell's viper venom denatured sample.

### (4) Experiment Methods

Prepare a SDS-PAGE gel and place it into the Hoefer™ electrophoresis unit. First fill the unit with the electrophoresis buffer, and then inject the processed sample onto the gel and place the setup in a cold room (4° C.). Adjust the power supply voltage to 150V-170V and the current to 100 mA, and start electrophoresis for 2 hours.

Prepare a PVDF membrane and mark it for easy identification. The sponge and blotting paper (Amersham Biosciences, UK) to be used were soaked in transfer buffer first and the PVDF to be used was sprayed with methanol. The materials were orderly placed into o the Hoefer™ transfer unit, and the unit were filled with buffer solution. Connect the power supply to the unit and adjust the voltage to 40V and the current to 120 milliamps. Power is applied and the setup was placed in a cold room overnight.

The transferred PVDF blot membrane was placed in 1X TBS with 10% skim milk powder solution to block for 30 minutes to 1 hour. Then dilute the primary antibodies 1:1000 and block for another 2 hours. The membrane was rinsed with TBST 3 times using SNAP i.d.™ Protein Detection System. Goat serum and secondary antibodies (5000 fold dilution) were sequentially added, and then the membrane was rinsed with TBST 3 times, after which chemical chromogenic reagents (AP buffer, NBT, BCIP) was reacted on the membrane. Finish by drying the membrane in an oven.

### Affinity Column

### (1) Experiment Materials

| | |
|---|---|
| Affinity column HiTrap | GE Healthcare, Cat. 17-0405-01 |
| Protein G HP | |
| Binding buffer (pH 7.0) | Independently prepared |
| Elution buffer (pH 3.0) | Independently prepared |
| PBS buffer | UniRegion Bio-Tech, Cat. UR-PBS001 |

### (2) Various Independently Prepared Solutions

### Binding Buffer (pH 7.0)

100 mM Sodium phosphate (J. T. Baker, Cat. 3828-01)
100 mM Sodium citrate (J. T. Baker, Cat. ADM873364)

### Elution Buffer (pH 3.0)

100 mM Sodium phosphate (J. T. Baker, Cat. 3828-01)
100 mM Sodium citrate (J. T. Baker, Cat. ADM873364)

### (3) Preparation of Experiment Materials

200 μL of the antibodies to be used was diluted 5 times with PBS to a total volume of 1000 μL for later use. The antigen (Russell's viper venom 2 μL) was diluted 250 times with PBS to a total volume of 500 μL.

### (4) Experiment Methods

Use the antibody purification liquid chromatography system to purify RVV-X. After washing with binding buffer (pH 7.0), the diluted antibodies was passed through the column, and then 500 μl of the antigen (diluted Russell's viper venom) to be purified was injected into the column. Elute with elution buffer (pH 3.0) and collect fractions of 3 ml in tubes, with a flow rate of 1 ml/minute.

### Freeze Drying

Using a vacuum freeze-drying technic, Russell's viper venom and RVV-X purified from it were powdered to maintain purity and to enable precise quantification.

### Clotting Time Test (Clotting Time Assay; CT)

### (1) Experiment Materials

| | |
|---|---|
| 0.11M Citric acid | Independently prepared |
| Tris buffer pH 8.3 | Independently prepared |
| Plasma | Peripheral venous blood from healthy |
| | humans |
| Russell's Viper Venom (RVV) | 20 ng/ml In 0.5% BSA/TBS |
| CaCl₂ Reagents | 0.2M CaCl₂ |

### (2) Preparation of Experiment Materials

Human blood and 0.11 M citric acid were mixed thoroughly in a ratio of 9:1, and plasma was separate from the mixture for later use by applying a centrifugal force of 5000 rpm. The plasma needs to be kept below 37° C. before use.

### (3) Experiment Methods

The method in O'Leary and Isbister (O'Leary M A, Isbister G K. (2010) A turbidimetric assay for the measurement of clotting times of procoagulant venoms in plasma. J Pharmacol Toxicol Methods. 61 (1): 27-31) and Isbister et al. (Isbister, G K, Woods, D., Alley, S., O'Leary, M A, Seldon, M., and Lincz, LF (2010). Endogenous thrombin potential as a novel method for the characterization of procoagulant snake venoms and the efficacy of antivenoms. Toxicon. 56:75-85) was adopted. The experiments were conducted with all materials kept under 37° C. using a spectrometer: uQuant MQX200 (Bio-Tek instruments, Inc., USA). Add 0.1 ml of diluted Russell's viper venom to the preheated plasma, then add 60 μl of CaCl₂, mix thoroughly, and record the absorbance value at 340 nm every 30 seconds for a total of 30 minutes. The lag time before the significant rise of absorbance value (representing coagulation) was the clotting time. The difference between the method used in the test group and the aforementioned method used in the control group was that to compare the inhibitory properties of the monoclonal antibodies across the same concentration, after adding Russell's viper venom to the plasma, the monoclonal antibodies to be tested were added to the plasma.

### Coagulation Factor X Activation Assay

### (1) Experiment Materials

| 0.11M citric acid | Independently prepared |
|---|---|
| Plasma | Peripheral venous blood from healthy |
| | humans |
| RVV-CaCl₂ mixture | Independently prepared |
| Tris buffer pH 8.3 | Independently prepared |
| Chromogenic substrate S- | Chromogenix, Milan, Italy Cat. |
| 2222 | 82031639 |

### (2) Various Independently Prepared Solutions

### Tris Buffer (pH 8.3)

50 mM Tris (J. T. Baker, Cat. 4109-6)
200 mM NaCl (J. T. Baker, Cat. 3624-05)
5 mM CaCl₂) (Merck, Cat. 102391)

### RVV-CaCl₂) Mixture

Russell's Viper Venom (RVV) 8 mg/100 ml
0.16 M CaCl₂) (Merck, Cat. 102391)
0.06 M NaCl (J. T. Baker, Cat. 3624-05)

### (3) Preparation of Experiment Materials

Human blood and 0.11 M citric acid were mixed thoroughly in a ratio of 9:1, and plasma was separate from the mixture for later use by applying a centrifugal force of 5000 rpm. The prepared S-2222 is first preheat by placing it in a water bath (37° C.).

### (4) Experiment Methods

The method in “Hematology: principles and procedures” by Barbara A. Brown was adopted. After the human plasma was diluted 20 times, take 0.1 ml and bath in 37° C. water for 5 minutes, then add 4.5 μl of RVV-CaCl₂ and bath it in 37° C. water bath for another minute. Lastly, 50 μl of preheated S-2222 was add and the absorbance value at OD₄₀₅ was measure and recording every 5 minutes for 1 hour. The difference between the method used in the test group and the aforementioned method used in the control group is that to test the effect of the monoclonal antibodies in inhibiting RVV activation of human coagulation factor X, after taking 0.1 ml of 20 times-diluted human plasma and before bathing in 37° C. water, different concentrations of monoclonal antibodies were added.

### Mouse IgG ELISA

### (1) Experiment Materials

| | |
|---|---|
| Monoclonal antibodies | Independently constructed |
| Mouse IgG ELISA Kit | Immunology Consultants Laboratory, Inc. |
| | Newberg, USA, Cat. E-90G |

### (2) Preparation of Experiment Materials

First, a standard concentration of IgG was diluted into a concentration of 600, 300, 150, 75, 37.5, 18.75, 9.375 ng/ml for later use according to the product manual. The monoclonal antibodies to be tested were serially diluted 50,000 times.

### (3) Experiment Methods

Mix 0.1 ml of each of the monoclonal antibodies to be tested and 0.1 ml of standard concentration solution on a 96 well plate, incubate at room temperature for 1 hour, rinse 3 times with a wash solution, and add 0.1 ml of enzyme-Ab conjugate. The plate was placed in the dark at room temperature for 30 minutes, then rinsed 3 times with the wash solution. Next, 0.1 ml of TMB substrate solution was added to the plate, and the plate was incubated at room temperature in the dark for 10 minutes. Stop the reaction with a stop solution, and use Enzyme immunoassay analyzer Dynex Technologies The Microtiter® company to detect the absorbance value at 450 nm and converts the reading to the original concentration.

### Median Lethal Dose Test (LD₅₀)

### (1) Experiment Materials

| | |
|---|---|
| Outbred mice | Tzu Chi University Animal Center |
| Russell's viper venom antiserum | Taiwan Centers for Disease Control |
| (horse polyclonal antibodies) | and Prevention |
| Russell's viper Venom | with freeze-dried Russell's viper venom |
| Plastic injection barrel with | BD, Taipei |
| needle | |
| 0.85% NaCl | Prepare your own |

### (2) Preparation of Experiment Materials

After weighing the freeze-dried Formosan Russell's viper venom powder, snake venom solutions of different concentrations were prepared with normal saline and prepared for intraperitoneal injection (IP). Mice of similar weight and age were selected for the experiment.

### (3) Experiment Methods

In accordance with the method of WHO guidelines for the production control and regulation of snake antivenom, after dividing the mice into groups (a group of 8, weighing about 21-25 g), the prepared snake venom solution with different concentrations of snake venom (i.e., the total weight of Russell's viper venom is different) was injected into the intraperitoneal cavity (IP) with a plastic syringe, and the mice were observed continuously for 48 hours. The survival rate of the mice was recorded, and the amount of snake venom used to achieve a 50% fatality rate was calculated.

### Median Thrombosis Dose Test (TD₅₀)

### (1) Experiment Methods

Mice weighing about 21-25 g were divided into groups each consisting of 4 mice, and different concentrations of snake venom were injected subcutaneously into the hind paw with a plastic syringe. Observe continuously for 72 hours, record the survival rate of the mice and the blackening of the paw, and then calculate the amount of snake venom used when 50% of the paws turned black.

### Antibodies Median Effective Dose (ED₅₀)

### (1) Preparation of Experiment Materials

After the Russell's viper venom antiserum and Russell's viper venom monoclonal antibodies were diluted into different concentrations, they are mixed with either 5 times the Median Lethal Dose (LD₅₀) or 1 time or 5 times the Median Thrombosis Dose (TD₅₀) according to the experimental protocol. The mixture were evenly mixed without centrifugation, and incubated at 37° C. for 30 minutes before use.

### (2) Experiment Methods

Challenge Method 1: Mice weighing about 21-25 g were divided into groups each consisting of 4 mice. The 5 times LD₅₀ snake venom combining various of antibodies combinations were injected into the intraperitoneal cavity (IP) of the mice with a plastic syringe. Observe continuously for 48 hours, and record the mouse survival rate. The aforementioned antibodies combinations were different dosage of the monoclonal antibodies, different dosage of the polyclonal antibodies, and combinations of 1 μg monoclonal antibodies with 1 mg-10 μg of polyclonal antibodies.

Challenge Method 2: Divide the mice weighing about 21-25 g into groups each consisting of 4 mice, inject subcutaneously 1 time TD₅₀ snake venom combining different dosage of monoclonal antibodies or different dosage of polyclonal antibodies into the paw with a plastic syringe. Observe continuously for 48 hours, record the survival of the mice and the blackening of the paw. In addition, Russell's viper venom antiserum (1 mg) was first injected into the abdominal cavity of mice, and after waiting for 3 hours, mix different doses (5 μg-1 μg) of monoclonal antibodies with 5 times TD₅₀ snake venom (250 μg) and inject subcutaneously into the paw. Observe continuously for 48 hours, record the survival of the mice and the blackening of the paw.

### Monoclonal Antibodies Sequencing

Use Trizol reagent (Invitrogen) to extract total RNA from hybridoma cells according to the instructions of the reagent, and then use the NucleoTrap mRNA Mini Kit (Macherey-Nagel GmbH & Co. KG.) to isolate mRNA. Purified mRNA was reverse transcribed using the ThermoScript RT-PCR system (Invitrogen) with oligo (dT) primers. The cDNA obtained by reverse transcription was cloned and amplified by PCR and TA kit (Promega), and the heavy chain and light chain variable region sequences was sequenced. Finally, the complementarity determining region (CDR) and framework region (FW) sequences in the variable region sequence were interpreted with reference to Kabat and ImMunoGeneTics databases.

### Example 1: Establishing of Russell's Viper Venom Mouse Model

In order to analyze and quantify Formosan Russell's viper venom and its antidote, a mouse model of Formosan Russell's viper venom was established with reference to the WHO guidelines.

### Example 1.1: Confirmation of Median Lethal Dose (LD₅₀)

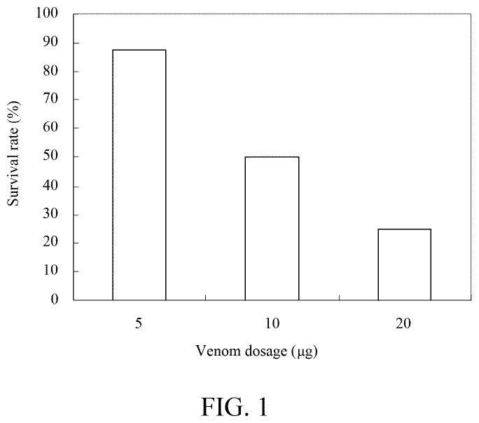

The mice were divided into groups (strain: ICR, age: 20-23 weeks, weight: 21-25 g), and intraperitoneally injected with different pre-formulated concentrations of Russell's viper venom. As shown in FIG. 1, when the total amount of snake venom reached 10 μg/25 g, the mortality rate of mice reached 50%. Hence, the Median Lethal Dose for mice was defined as 10 μg/25 g.

### Example 1.2: Determination of Median Thrombosis Dose (TD₅₀)

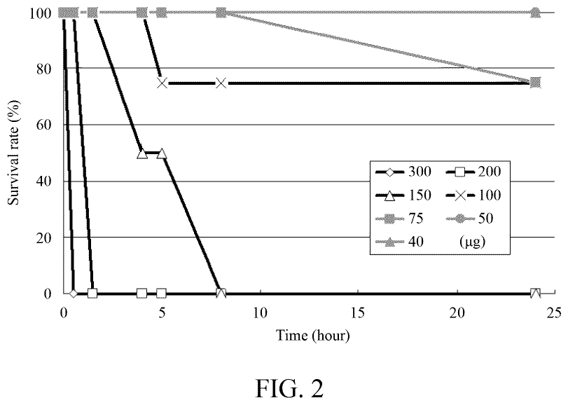

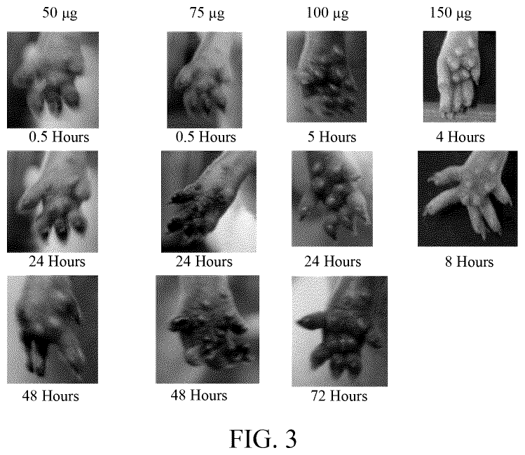

Russell's viper venom had both hemotoxins and neurotoxins. While neurotoxicity occurred quickly in envenomed small animals, Russell's viper venom was mainly toxic to large animals through hemotoxins. Therefore, in order to accurately evaluate the effect of hemotoxins in mice, the paws of mice (strain: ICR, age: 20-23 weeks, weight: 21-25 g) were selected as the test site. Pre-formulated Russell's viper venom was injected subcutaneously into the paws of mice, and changes in the paws was observed to see if there were blood clots and thrombosis. Experimental results (FIGS. 2 and 3) showed that injecting a total amount of 40 μg snake venom will not cause blood clots or thrombosis in mice. Injecting a total amount of 50 μg of snake venom caused the paws of mice to start showing thrombosis after 30 minutes.

After about 1 hour, all mice had half of their paws completely blackened. After 1 day of observation, the paws that were thrombosed began to dry up due to tissue necrosis. After 2 days of observation, the dried paws broke off due to tissue necrosis. Injection of a total amount of 75 μg of snake venom began to be fatal for the mice. Injection of a total amount of 100 μg of snake venom or more caused the mice to die before the paws of the mice started to blacken. In summary, the Median Thrombosis Dose of Russell's viper venom in mice was defined as 50 μg/25 g.

### Example 2: Preparation of Monoclonal Antibodies Against Russell's Viper Venom

BALB/c female mice aged 6 to 8 weeks were injected and immunized intraperitoneally with an injection containing 0.1×LD 50 of Formosan Russell's viper venom and an equal volume of Freund's complete adjuvant. Thereafter, three additional immunization injections were given, each two weeks apart, with the adjuvant of the additional injections changed to Freund's incomplete adjuvant. After four immunizations, orbital blood was drawn, and the titer of Russell's viper venom protein antibodies in the serum was determined by enzyme-linked immunosorbent assay (ELISA). The spleen cells of the mice with high titers were removed using sterile techniques and fused with NS-1 myeloma cells in a solution containing PEG. After screening in a culture medium containing HAT, immunofluorescence assay was used to select the hybridoma cells that were able to secrete Russell's viper venom antibodies. Through the use of a limiting dilution method, a single cell was cultured into a single cell line, and the enzyme-linked immunosorbent assay was used again to identify hybridoma cell lines that could secrete monoclonal antibodies that recognized Russell's viper venom. A total of 18 monoclonal antibodies against Russell's viper venom were isolated.

### Example 3: Test for Specific Recognition of Russell's Viper Venom Factors by Monoclonal Antibodies

In order to test the ability of the 18 monoclonal antibodies established in Example 2 in recognizing Russell's viper venom, a Western blot method was used to identify the ability of each monoclonal antibody to bind to Formosan Russell's viper venom. The results showed that all 18 monoclonal antibodies were able to bind to an approximately 95 kDa protein molecule in the Formosan Russell's viper venom. This showed that all 18 monoclonal antibodies established in Example 2 were able to specifically recognize the protein molecules of the Formosan Russell's viper venom.

Amongst the known Russell's viper venom factors, the native protein molecules of Russell's viper venom factor-X (RVV-X) across various subspecies had an approximately molecular mass of 92 to 95 kDa. Hence, the antigen identified by the monoclonal antibodies molecule might be RVV-X.

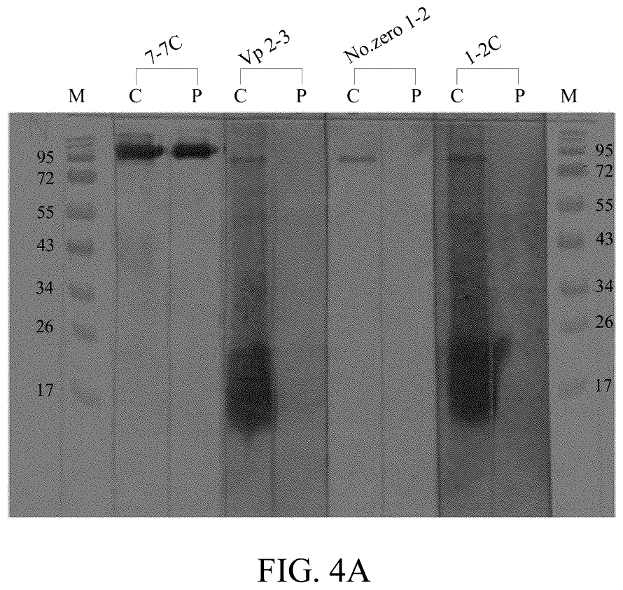

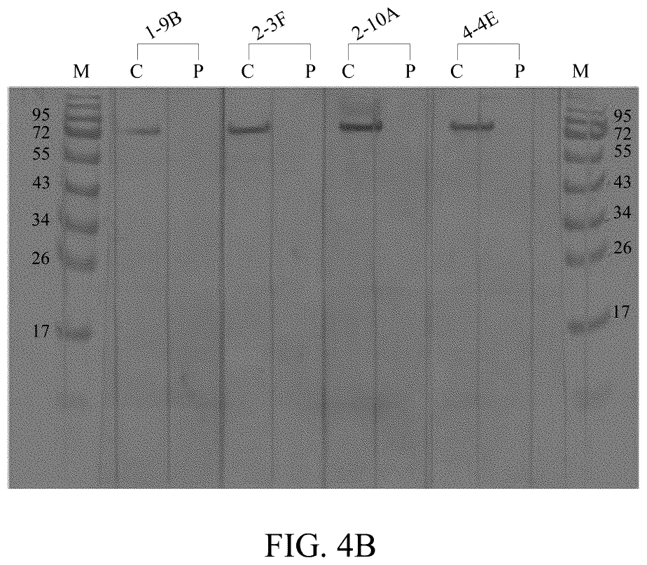

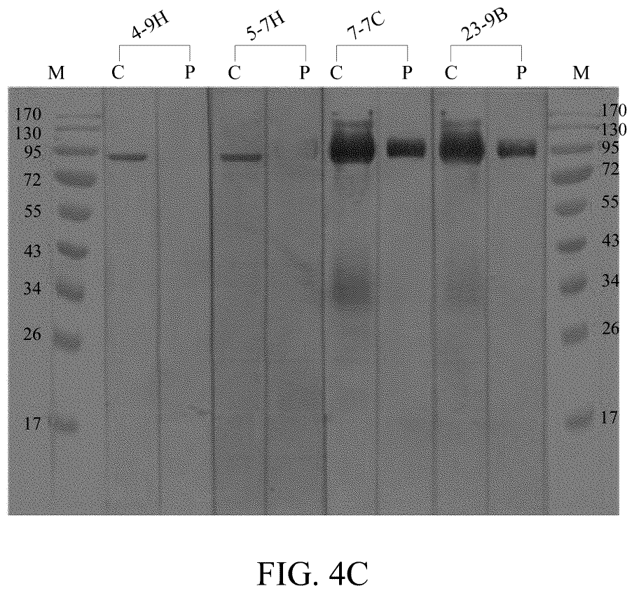

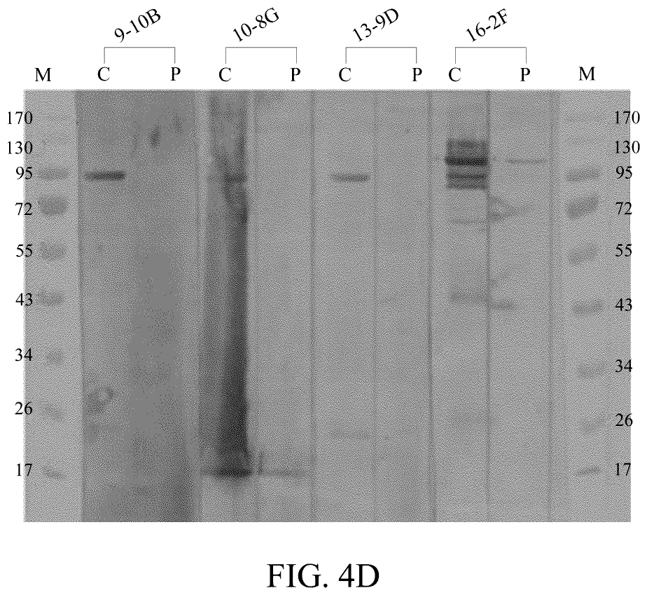

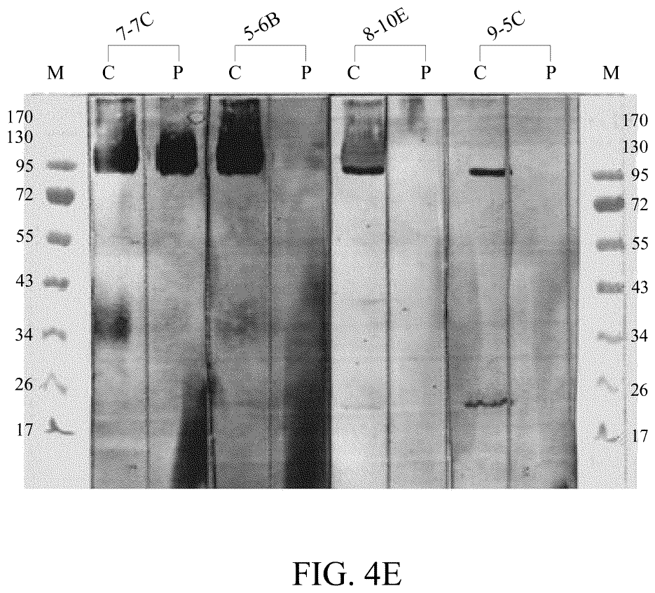

Therefore, pure RVV-X protein derived from purified Daboia russelii russelii (i.e. Indian Russell's viper) venom was purchased (RVV-X Activator; Haematologic Technologies Inc.). Further, a Western blot method was used to confirm the antibody ability to bind to pure RVV-X protein molecules for the 18 monoclonal antibodies established in Example 2. The results showed that only 4 monoclonal antibodies, 5-6B, 7-7C, 16-2F and 23-9B, could recognize the approximately 95 kDa protein molecules in pure RVV-X. In addition, 16-2F also recognized an additional protein molecule of approximately 100 kDa in pure RVV-X. The above Western blot test results were shown in FIGS. 4A-4E, in which column C represented the Formosan Russell's viper venom, and column P represented the purchased pure RVV-X protein.

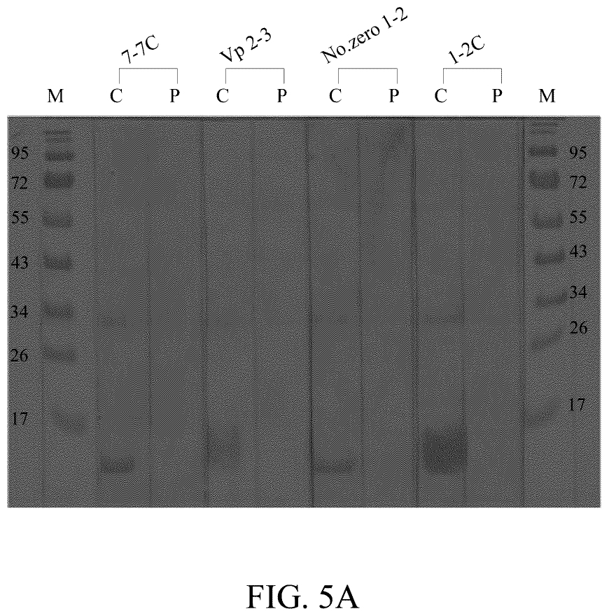

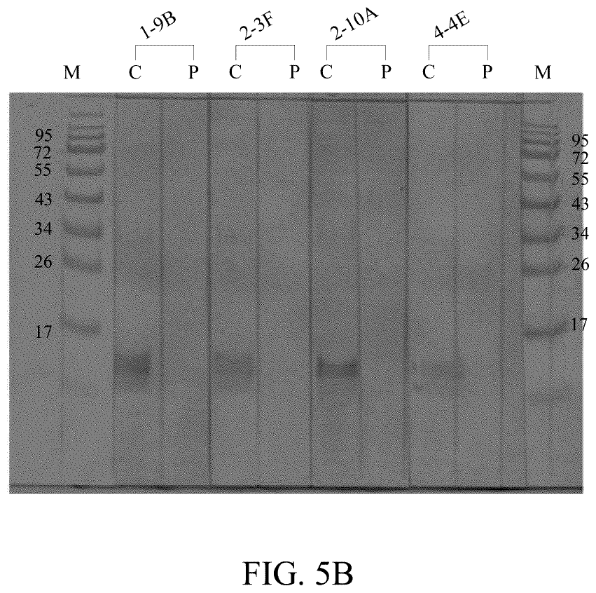

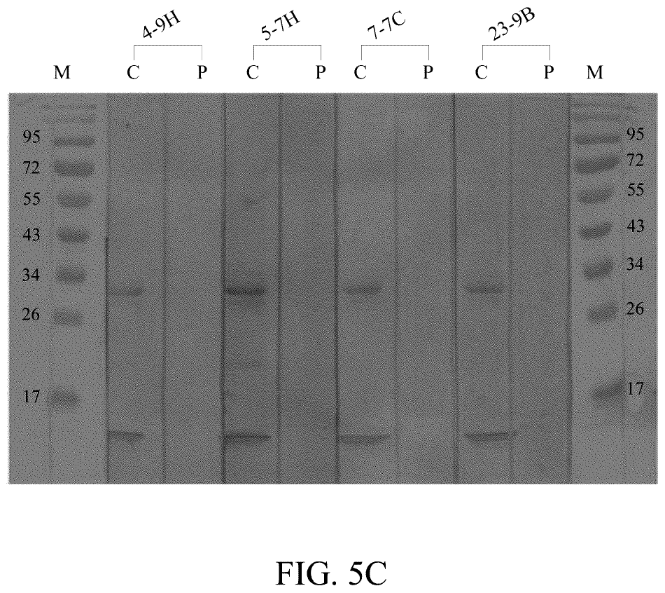

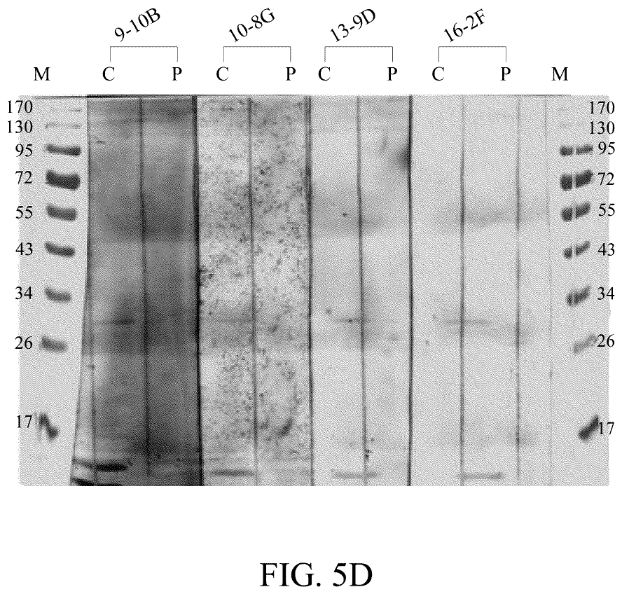

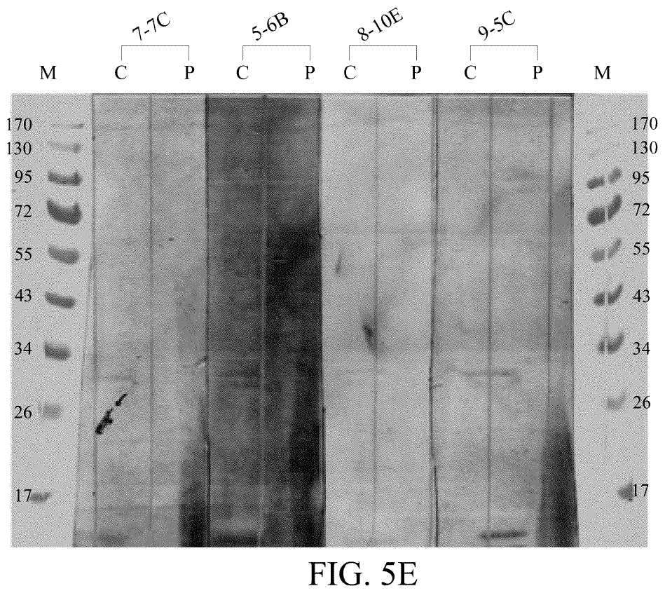

The aforementioned Formosan Russell's viper venom and pure RVV-X were further reduced and denatured, and then the denatured Formosan Russell's viper venom and denatured pure RVV-X were individually subjected to Western blotting using the 18 monoclonal antibodies of Example 2. As shown in FIGS. 5A-5E, the 18 monoclonal antibodies bound to a 32 kDa protein and a less than 17 kDa protein in the denatured Formosan Russell's viper venom, but did not recognize the denatured pure RVV-X. It was inferred from the above experimental results that the monoclonal antibodies in the monoclonal antibody library recognized RVV-X's conformational structure epitope within the three-dimensional space rather than linear structure epitope. In FIGS. 5A-5E, column C represented Formosan Russell's viper venom, and column P represented purchased pure RVV-X protein.

### Example 4: Monoclonal Antibody Specifically Recognized the Russell's Viper Venom Factors of Formosan Russell's Viper

Compared with the purchased pure RVV-X protein derived from the Indian Russell's viper, the Formosan Russell's viper was another species of Russell's viper and was classified as Daboia siamensi. Hence, its venom factors might have different protein sequence. Therefore, in order to confirm whether the RVV-X monoclonal antibody identified in Example 3 could recognize the RVV-X molecule of the Formosan Russell's viper, the RVV-X molecules of the Formosan Russell's viper were first purified, and then the ability of RVV-X monoclonal antibody to recognize Formosan Russell's viper RVV-X was tested.

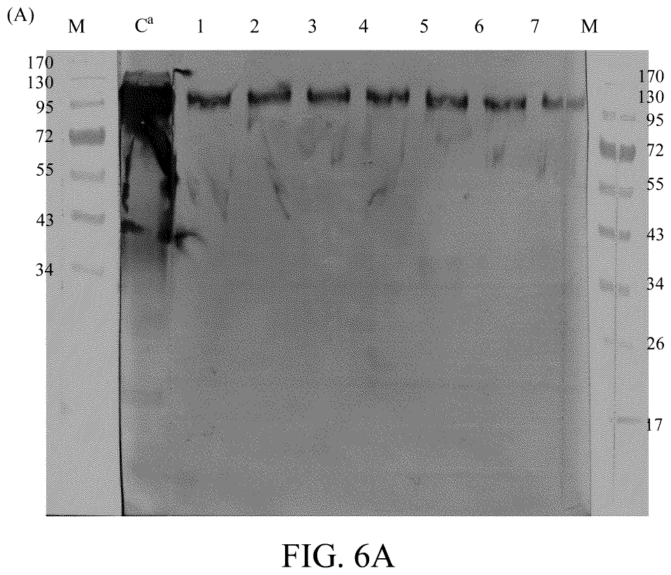

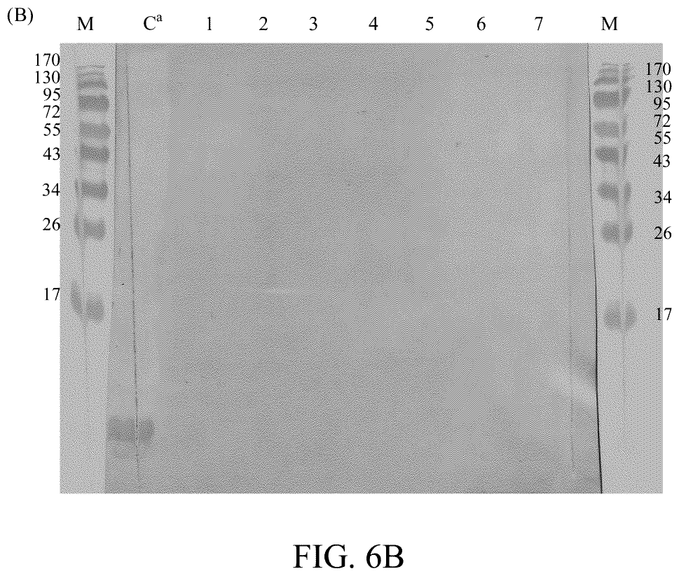

First, in order to purify Formosan Russell's viper RVV-X, an affinity column was used. The affinity column was prepared by attaching the monoclonal antibody 23-9B that recognized RVV-X to the affinity column to produce an affinity column for RVV-X. Next, the prepared affinity column was used to perform column chromatography of the Formosan Russell's viper venom by letting the monoclonal antibody to bind to RVV-X in the snake venom, and then eluting the affinity column. The elution was checked by Western blotting using the monoclonal antibody 7-7C. As shown in FIG. 6A, after column purification, a 95 kDa snake venom protein molecule was identifiable in the elution. The results showed that the Formosan Russell's viper RVV-X was successfully purified using affinity column chromatography, and also showed that the disclosed monoclonal antibodies could recognize the Formosan Russell's viper RVV-X. However, as shown in FIG. 6B, if the elution was reduced and denatured, no protein molecules could be identified. This result corresponded with that of Example 3.

The above results showed that the RVV-X monoclonal antibody identified in Example 3 could specifically recognize RVV-X of all Russell's viper including Daboia russelii and Daboia siamensis.

### Example 5: Analysis of the Potency of Monoclonal Antibodies in Neutralizing Russell's Viper Venom In Vitro

In order to evaluate the neutralizing potency of the monoclonal antibodies 5-6B, 7-7C, 16-2F and 23-9B, biochemical experiments relating to coagulation mechanism were used for confirmation.

### Example 5.1: The Monoclonal Antibodies could Inhibit Russell's Viper Venom-Induced Clotting Time Acceleration

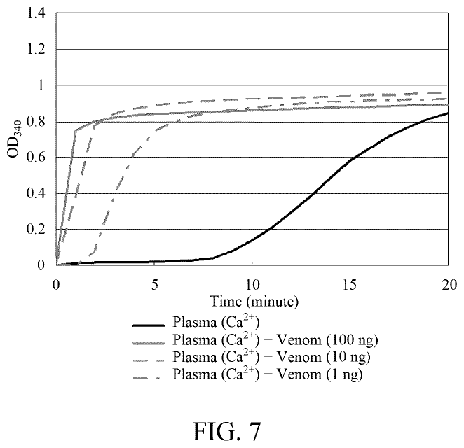

The RVV-X in Russell's viper venom activated human coagulation factor X and caused clotting. First, it was confirmed that the blood clotting speed in in vitro blood clotting method can be affected by Russell's viper venom. Then, the effect of monoclonal antibodies in neutralizing Russell's viper venom was test. When blood coagulated, the absorption value at 340 nm would rise rapidly due to plasma coagulation. This time point was defined as the clotting time (CT), which could be used to determine the clotting speed. The experiments found that adding different concentrations of Russell's viper venom to human recalcified plasma visibly shorten the clotting time, and the higher the dose, the shorter the clotting time (FIG. 7).

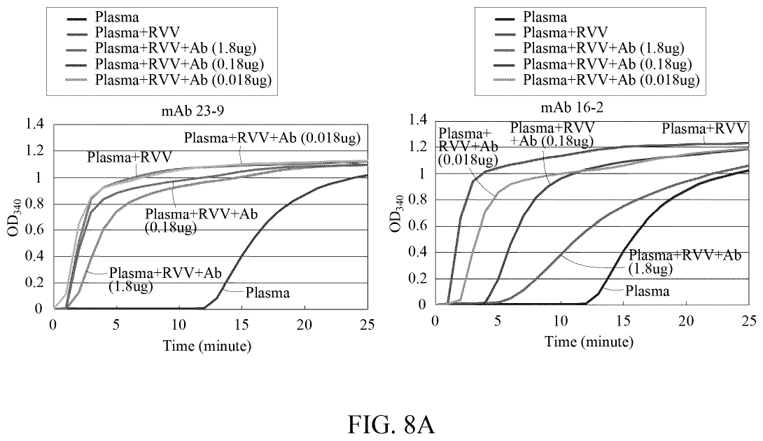

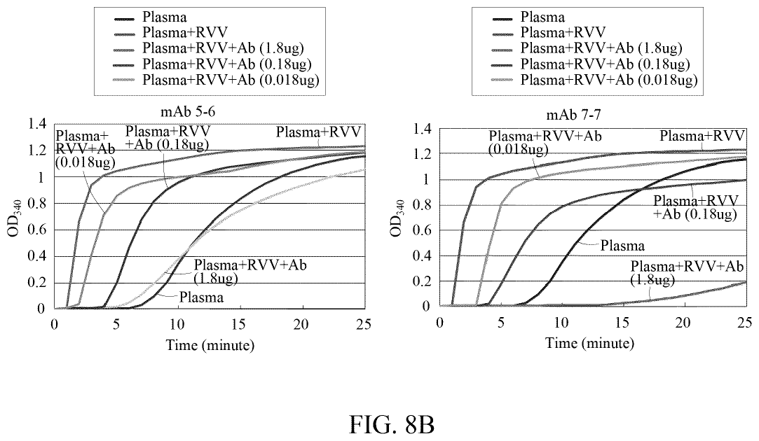

An experiment setup of 1 ng of Russell's viper venom was selected to test the monoclonal antibodies inhibitory potency on the shortening of clotting time caused by Russell's viper venom. Monoclonal antibodies 5-6B, 7-7C, 16-2F and 23-9B at a dosage between 18 ng to 1800 ng was add to the Russell's viper venom spiked recalcified plasma for reaction. The experiment found that adding different doses of monoclonal antibodies 5-6B, 7-7C, 16-2F had a visible inhibitory effect on clotting time, but monoclonal antibody 23-9B had no such inhibitory effect (FIGS. 8A and 8B).

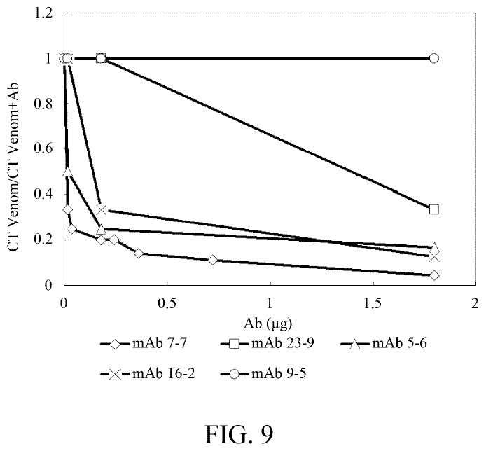

The ratio obtained from dividing the clotting time after adding Russell's viper venom (CT_{snake venom}) by the clotting time after adding Russell's viper venom and monoclonal antibody (CT_{snake venom+Ab}) indicated the potency of the antibody. The smaller the ratio, the more potent the monoclonal antibody in preventing coagulation caused by Russell's viper venom (FIG. 9). For example, when 18 ng of monoclonal antibody was added, 7-7C had the lowest ratio (CT_{snake venom}/CT_{snake venom+Ab}), indicating that 7-7C had the best neutralization effect. To achieve the same neutralizing effect, the other 3 monoclonal antibodies required a higher dosage (5-6B: about 30 ng, 16-2F: 180 ng and 23-9B: 1800 ng).

### Example 5.2: The Monoclonal Antibodies were Able to Neutralize Russell's Viper Venom-Induced Activation of Coagulation Factor X (Coagulation Factor X Activation Assay)

In the clotting cascade, the activation of coagulation factor X was a key step in the cascade. The activated coagulation factor X (i.e. coagulation factor Xa) cleaved prothrombin to thrombin, which in turn caused blood clotting. RVV-X in Russell's viper venom also affected the blood clotting pathway through the activation of this factor.

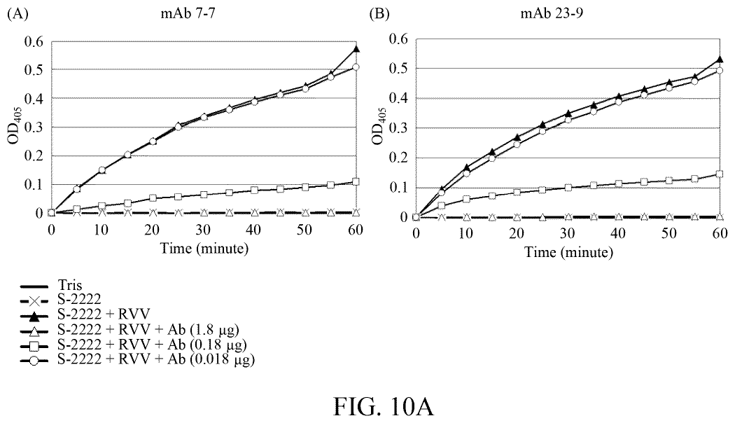

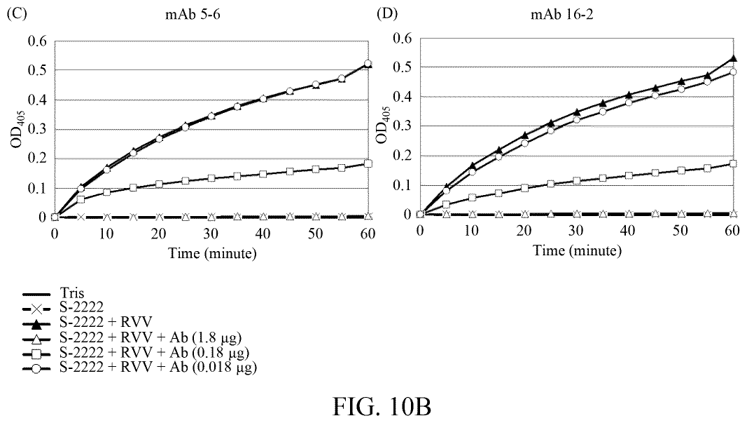

S-2222 was a compound synthesized by imitating prothrombin with an coagulation factor Xa enzyme cleavage site. S-2222 carried a chromogenic substance and the chromogenic substance turned yellow for easy detection once cleaved by coagulation factor Xa. Hence, S-2222 could determine whether Russell's viper venom activate coagulation factor X. Further, the neutralization effect of monoclonal antibodies 5-6B, 7-7C, 16-2F and 23-9B of the present disclosure could be tested through this method. As shown in FIGS. 10A and 10B, Russell's viper venom did activate coagulation factor X (see ▴ in FIG. 10). After adding 18 ng to 1800 ng of the antibodies, the activation of the coagulation factor X to coagulation factor Xa by Russel's viper venom were effectively inhibited, and as the antibody dosage increased, the inhibition strengthened, even achieving complete inhibition. However, there was no significant difference in the inhibitory effect amongst the monoclonal antibodies.

Based on the above results, the monoclonal antibodies of the present disclosure were able to neutralize the Russell's viper hemotoxins by, for example, but not limited to, blocking the of Russell's viper venom coagulation factor X activation pathway, thereby inhibiting the coagulation reaction caused by the hemotoxins.

### Example 6: Analysis of Monoclonal and Polyclonal Antibodies Neutralization Dosage (ED₅₀) of Russell's Viper Venom Factor

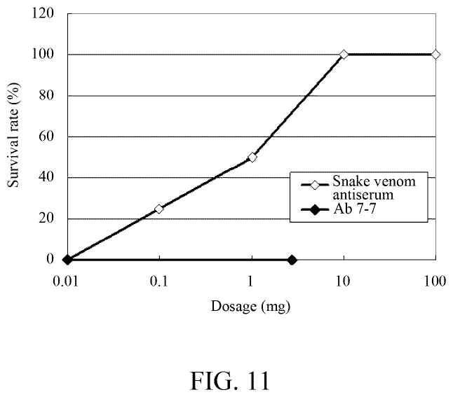

Two challenge methods were used to evaluate the neutralization dosage and potency of the antibodies. The first was intraperitoneal injection of 5 times the Median Lethal Dose (50 μg/25 g) of Russell's viper venom. In this method, monoclonal antibody 7-7C or Russell's viper venom antiserum (horse polyclonal antibody serum, Taiwan Centers for Disease Control and Prevention) was injected simultaneously with the Russell's viper venom during the envenomation challenge, and the death of mice was recorded 48 hours later. As shown in FIG. 11, the intraperitoneal injection of Russell's viper venom antiserum could effectively neutralize Russell's viper venom-induced mice death, and the neutralization effect increased with dose. The median neutralization dosage ED₅₀ is 1 mg/25 g. In contrast, when mice receive intraperitoneal injection of monoclonal antibody 7-7C, even when the dosage reached approximately 3 times of the ED₅₀ of Russell's viper venom antiserum (2.7 mg/25 g), the monoclonal antibody 7-7C was still unable to neutralize the fatality of snake venom. The result showed that the monoclonal antibody 7-7C had no protective effect against the lethal toxicity produced by intraperitoneal injection of Russell's viper venom in mice.

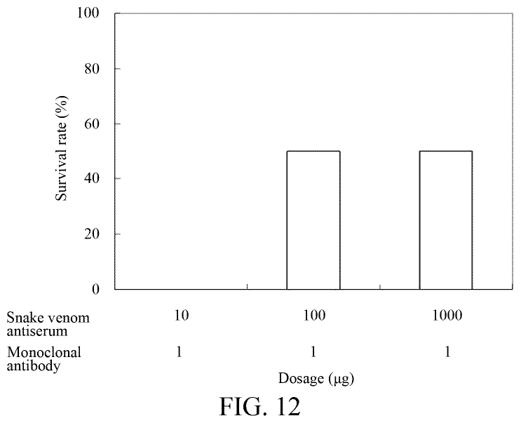

Whether the neutralization potency of the monoclonal antibody 7-7C and the Russell's viper venom antiserum has an synergistic effect was further tested. The same method of intraperitoneal injection of 5 times the Median Lethal Dose was adopted, but the monoclonal antibody 7-7C and the Russell's viper venom antiserum were mixed in different proportions and injected simultaneously with the Russell's viper venom during the envenomation challenge. The venom neutralization effect of the different monoclonal antibody and antiserum mixture was recorded. As shown in FIG. 12, when a dosage of 10, 100 and 1000 μg of Russell's viper venom antiserum were mixed with 1 μg of monoclonal antibody 7-7C, the ED₅₀ dosage of Russell's viper venom antiserum was reduced to 100 μg. The reduced ED₅₀ was only 1/10 of the ED₅₀ when Russell's viper venom antiserum was used alone. As demonstrated by the results, the monoclonal antibody 7-7C and the Russell's viper venom antiserum had visible synergistic detoxification effects.

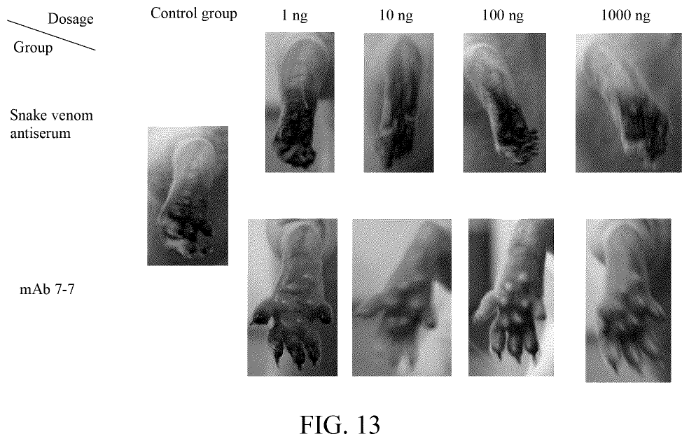

The second method of envenomation challenge was paw injection. First, a Median Thrombosis Dose (TD₅₀, 50 μg/25 g) of Russell's viper venom was mixed with monoclonal antibody 7-7C or Russell's viper venom antiserum and injected into the paws of mice. The blackening of the paw was recorded 48 hours later. As shown in FIG. 13, a dosage of 1 ng/25 g of the monoclonal antibody 7-7C could effectively inhibit the blackening of paw caused by Russell's viper venom, and a dosage of 10 ng/25 g could completely neutralize the coagulation toxicity of the snake venom. In contrast, even when the dosage of Russell's viper venom antiserum reached 1000 ng/25 g, there were no inhibitory effect on the blackening of the paws. As demonstrated by the results, the effectiveness of the Russell's viper venom antiserum in neutralizing the coagulation toxicity caused by Russell's viper venom was far less than that of the monoclonal antibody 7-7C.

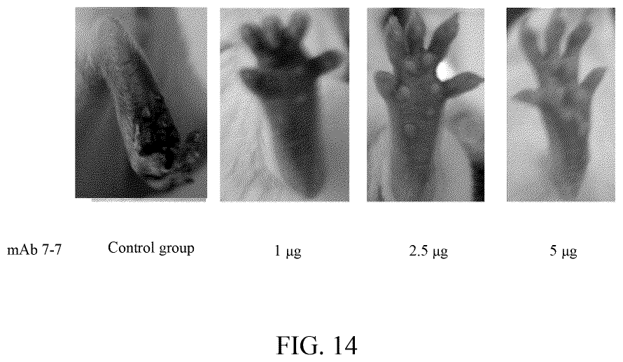

In order to further test the detoxification effect of the monoclonal antibody 7-7C on the coagulation toxicity caused by a high dosage of Russell's viper venom, it was necessary to overcome the fact that injection of more than 75 μg of Russell's viper venom into the paw was lethal to mice. Therefore, 1 mg/25 g of the Russell's viper venom antiserum was first intraperitoneally injected into the test mice, and then the mice were injected with 250 μg/25 g (5×TD₅₀) of Russell's viper venom into the paw, in the hope that the clotting conditions of envenomed mice and the neutralizing effect of the monoclonal antibody could be observed under high venom dosage. As shown in FIG. 14, unlike in Example 1.2, the mice would not die before their paw started to blacken when a total amount of 100 μg or more Russell's viper venom was injected under this experiment setup. This result indicated that the aforementioned result of the monoclonal antibody 7-7C having no protective effect against the lethal toxicity produced by intraperitoneal injection of Russell's viper venom in mice was because the monoclonal antibody 7-7C could not protect mice from acute neurotoxins toxicity. Under this experiment setup, injecting 2.5 μg of the monoclonal antibody 7-7C into the paw was able to relieve the thrombosis in the paw of mice after 48 hours.

### Example 7: Monoclonal Antibody Sequencing

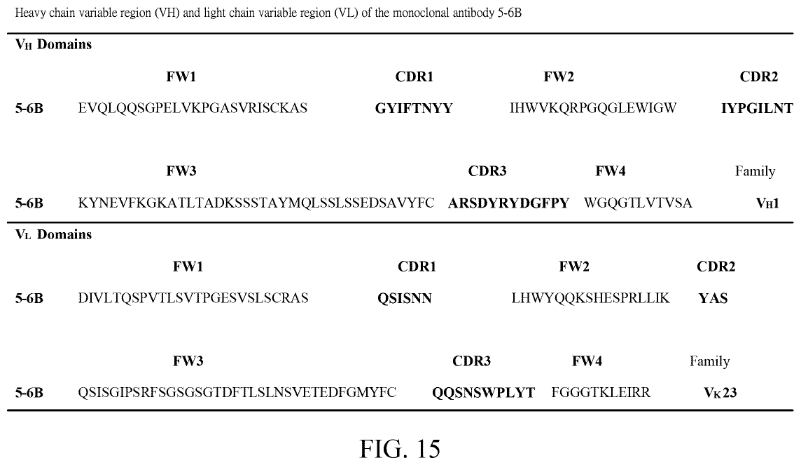

The sequencing result of monoclonal antibody 5-6B were shown in FIG. 15. The amino acid sequence of its heavy chain variable region was EVQLQQSGPELVKPGASVRISCKASGYIFTNYYIHWVKQRPGQGLE WIGWIYPGILNTKYNEVFKGKATLTADKSSSTAYMQLSSLSSEDSAV YFCARSDYRYDGFPYWGQGTLVTVSA (SEQ ID NO: 1), and the amino acid sequence of its light chain variable region was

(SEQ ID NO: 2)
DIVLTQSPVTLSVTPGESVSLSCRASQSISNNLHWYQQKSHESPRLL
IKYASQSISGIPSRFSGSGSGTDFTLSLNSVETEDFGMYFCQQSNSW
PLYTFGGGTKLEIRR.

Further, monoclonal antibody 5-6B HCDR1 amino acid sequence was GYIFTNYY (SEQ ID NO: 3); HCDR2 amino acid sequence was IYPGILNT (SEQ ID NO: 4); and HCDR3 amino acid sequence was ARSDYRYDGFPY (SEQ ID NO: 5). Monoclonal antibody 5-6B LCDR1 amino acid sequence was QSISNN (SEQ ID NO: 6); LCDR2 amino acid sequence was YAS (SEQ ID NO: 7); and LCDR3 amino acid sequence was QQSNSWPLYT (SEQ ID NO: 8).

### CONCLUSION

The experiment results found 18 monoclonal antibodies that was able to recognize Russell's viper venom, wherein the Russell's viper venom factor that the monoclonal antibodies 7-7C, 5-6B, 16-2F and 23-9B could specifically bind to was RVV-X. The monoclonal antibodies were able to specifically bind to RVV-X of both Daboia russelii and Daboia siamensis. The monoclonal antibodies could not recognize denatured RVV-X, which indicated that the monoclonal antibodies recognize the conformational structure epitope.

During clotting, various coagulation factors were activated through a cascade reaction. Further, the activation of coagulation factor X into coagulation factor Xa was a shared pathway of the intrinsic and extrinsic clotting pathways. RVV-X in Russell's viper venom had the ability to directly activate coagulation factor X into coagulation factor Xa. Russell's viper venom directly activated clotting pathway, thereby causing clotting, thrombosis, and even systemic hemorrhage due to excessive depletion of blood coagulation factors. After confirming that Russell's viper venom could shorten the clotting time, the experiments found that the monoclonal antibodies 5-6B, 7-7C and 16-2F were able to inhibit clotting caused by the snake venom, but the monoclonal antibody 23-9B had no inhibitory effect. In addition, through further analysis of coagulation factor X activation using S-2222, it was confirmed that the antagonism between Russell's viper venom and the aforementioned monoclonal antibodies indeed acted upstream of coagulation factor Xa.

Through the mouse paw envenomation experiment, it was found that purified RVV-X alone could not cause the blackening of the mouse paw, nor was it fatal to the mice. Therefore, in the mice paw envenomation experiments, the mice must be challenged with Russell's viper venom. However, Russell's viper venom was a snake venom that contained both hemotoxins and neurotoxins. Neurotoxins had a rapid and visible effect on small animals, while only hemotoxins can exert a venomous effect on large animals. Therefore, when constructing an animal model of Russell's viper venom using mice, intraperitoneal envenomation would make the effect of the neurotoxins (death in about 12 hours) stood out. Meanwhile, although envenomation of the paws, which was far away from the torso, was able to cause thrombosis and necrosis of the paw tissue, the neurotoxins still exerted its effects and led to mice death when the envenomation dosage was too high. Since the monoclonal antibodies disclosed in the present disclosure mainly neutralize hemotoxins, injecting the monoclonal antibodies under these two experiment setup would not produce a protective effect. This was also the limiting factor that forbade the mouse thrombosis model to be challenged with 5 times TD₅₀. In this regard, it was assumed that Russell's viper venom antiserum (snake venom horse antiserum) was able to neutralize the neurotoxins of Russell's viper venom and to prevent the death of mice. Therefore, the experiment of the present disclosure tried to first inject into the abdominal cavity of test mice with Russell's viper venom antiserum, and found that at the same 5 times Median Thrombosis Dose (5×TD₅₀) of 250 μg, the neurotoxins were effectively neutralized and the fatalities of mice were improved. Under this experiment setup, after injecting 250 μg of snake venom into the paw, the control group that was not administered with the monoclonal antibody 7-7C would still exhibit blackening of the paws, while the monoclonal antibody injection was able to effectively treat the blackening of paws. The results indicated that the monoclonal antibodies were able to effectively neutralize the Russell's viper's hemotoxins, while the Russell's viper venom antiserum primarily neutralized the Russell's viper's neurotoxins that killed the mice.

Because of the size of a human being, the main venomous effects were exerted by hemotoxins in a patient envenomed by a Russell's viper. Therefore, to treat the patient envenomed by a Russell's viper, the results of the embodiments of the present disclosure showed that: the monoclonal antibodies of the present disclosure could neutralize hemotoxins, especially RVV-X, and were effective in preventing systemic hemorrhage, extensive thrombosis, and renal failure. The effectiveness of the monoclonal antibodies of the present disclosure was higher than the existing Russell's viper venom antiserum, and a higher synergistic neutralization benefits could be obtained by the combined usage of the two, including, but not limited to, reducing the dosage of Russell's viper venom antiserum needed.

Although some embodiments of the present disclosure have been illustrated above in detail, those skilled in the art can make various modifications and variations to the illustrated embodiments without departing from the teachings and advantages of the present disclosure. Therefore, such modifications and variations should be encompassed within the scope of the present disclosure as defined in the appended claims.

What is claimed is:

1. An antivenom monoclonal antibody of Russell's viper or an antigen-binding moiety thereof, wherein the monoclonal antibody or the antigen-binding moiety thereof specifically binds to a Russell's viper venom factor-V (RVV-V) or a Russell's viper venom factor-X (RVV-X).

2. The monoclonal antibody or the antigen-binding moiety thereof of claim 1, wherein the monoclonal antibody or antigen-binding moieties thereof specifically binds to RVV-X.

3. The monoclonal antibody or the antigen-binding moiety thereof of claim 1, wherein the monoclonal antibody or the antigen-binding moiety thereof comprises a heavy chain variable region (VH) comprising three heavy chain complementarity determining regions (HCDR) and a light chain variable region (VL) comprising three light chain complementarity determining regions (LCDR).

4. The monoclonal antibody or the antigen-binding moiety thereof of claim 3, wherein the VH comprises an HCDR1 including an amino acid sequence of SEQ ID NO: 3, an HCDR2 including an amino acid sequence of SEQ ID NO: 4, and an HCDR3 including an amino acid sequence of SEQ ID NO: 5; and wherein the VL comprises an LCDR1 including an amino acid sequence of SEQ ID NO: 6, an LCDR2 including an amino acid sequence of SEQ ID NO: 7, and an LCDR3 including an amino acid sequence of SEQ ID NO: 8.

5. The monoclonal antibody or the antigen-binding moiety thereof of claim 3, wherein the VH comprises an amino acid sequence of SEQ ID NO: 1 or an amino acid sequence at least 85%, 90% or 95% identical to SEQ ID NO: 1.

6. The monoclonal antibody or the antigen-binding moiety thereof of claim 3, wherein the VL comprises an amino acid sequence of SEQ ID NO: 2 or an amino acid sequence at least 85%, 90% or 95% identical to SEQ ID NO: 2.

7. The monoclonal antibody or the antigen-binding moiety thereof of claim 3, wherein the VH comprises an amino acid sequence of SEQ ID NO: 1, and the VL comprises an amino acid sequence of SEQ ID NO: 2.

8. A method of treating envenomation of snake venom of Russell's viper in a subject in need thereof, comprising administering an effective amount of the monoclonal antibody or the antigen-binding moiety thereof according to claim 1 to the subject.

9. The method of claim 8, wherein the effective amount is in a range from 1 ng to 10 μg.

10. The method of claim 8, wherein the effective amount is in a range from 18 ng to 1800 ng.

11. The method of claim 8, wherein the effective amount is in a range from 1 μg to 5 μg.

12. The method of claim 8, wherein administration of the monoclonal antibody or the antigen-binding moiety thereof is intraperitoneal, intramuscular, intravenous or subcutaneous.

13. The method of claim 8, further comprising a pharmaceutical acceptable excipient.

14. The method of claim 8, wherein the monoclonal antibody or the antigen-binding moiety thereof is administered in combination with an additional antidote for Russell's viper.

15. A kit for determining a concentration of venom of Russell's viper, comprising:
the monoclonal antibody or the antigen-binding moiety thereof according to claim 1;
a vessel; and
an instruction for using the monoclonal antibody or the antigen-binding moiety thereof to determine the concentration of venom of Russell's viper.
